(19) United States
(12) Patent Application Publication
(10) Pub. No.: US 2004/0161468 A1
(43) Pub. Date: Aug. 19, 2004

(54) Stabilization of aromatic active principles using aromatic polymers

(75) Inventors: Leila Toumi, Sannois (FR); Anne-Laure Bernard, Courbevoie (FR); Henri Samain, Bievres (FR); Luc Nicolas Morgantini, Rully (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/726,557

(22) Filed: Dec. 4, 2003

Related U.S. Application Data
(60) Provisional application No. 60/459,642, filed on Apr. 3, 2003.

(30) Foreign Application Priority Data
Dec. 4, 2002 (FR) 0215297

(51) Int. Cl. A61K 7/06; A61K 9/14

(52) U.S. Cl. 424/489; 424/70.1; 424/401

(57) ABSTRACT

The invention relates to particles containing at least one active principle, which is preferably water-soluble, containing one or more aromatic, carbocyclic or heterocyclic, monocyclic or fused polycyclic groups, with a molecular weight of less than or equal to 1000, and at least one synthetic polymer containing aryl groups with a glass transition temperature (T_{g}) of greater than or equal to 45, to processes for preparing such particles and to compositions containing them.

### REFERENCE TO PRIOR APPLICATIONS

This application claims priority to U.S. provisional application 60/459,642 filed Apr. 3, 2003, and to French patent application 02 15297 filed Dec. 4, 2002, both incorporated herein by reference.

### FIELD OF THE INVENTION

The present invention relates to a process for protecting and/or stabilizing aromatic active principles by incorporating or encapsulating such an active principle into a synthetic polymer containing aryl groups, to particles obtained by such a process, and to compositions, especially cosmetic compositions, containing such particles.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

The stabilization of water-soluble active principles of low mass in pharmaceutical or cosmetic compositions containing a certain fraction of water is a recurrent problem. The encapsulation of active principles in standard microcapsules or nanocapsules—i.e. particles consisting of a hydrophilic liquid phase surrounded by a polymer shell—obtained either by interface polymerization or by water/oil/water multiple emulsion followed by evaporation of the solvent, or by coacervation, has been found in most cases to be unsatisfactory since the capsules prepared generally release the active principles more or less quickly into the outer aqueous phase. Such a release is also observed for other types of organized structures, such as vesicles.

Incorporation into solid polymer particles is also problematic since water-soluble active principles are generally incompatible with the polymer matrix and “sweating” is observed, i.e. a slow migration of the active principle molecules towards the external medium.

### DETAILED DESCRIPTION OF THE PREFERRED EMBODIMENTS

The inventors have solved these and other problems for a particular group of active principles of low mass, i.e. for active principles comprising one or more aromatic nuclei, by incorporating these active principles into particles comprising a polymer that bears a number of aryl groups and that is in vitreous form at room temperature.

The aromatic active principles enclosed in such a matrix or shell of vitreous aryl-containing polymer are not released into the surrounding aqueous phase and are fully protected against chemical or physicochemical agents liable to result in their degradation.

Consequently, one subject of the present invention is particles comprising:

at least one active principle, which is preferably water-soluble, comprising one or more aromatic, carbocyclic or heterocyclic, monocyclic or fused polycyclic groups, with a molecular weight of less than or equal to 1000, and

at least one synthetic polymer containing aryl groups, with a glass transition temperature (Tg) of greater than or equal to 45° C.

Preferably, the aryl groups of the synthetic polymer are phenyl or phenylene groups.

When the active agent is of heterocyclic nature, it is preferably a polycyclic structure comprising at least one benzene nucleus.

A subject of the invention is also a process for preparing such particles, in other words a process for protecting or stabilizing aromatic active principles of low mass by incorporating such an active principle into a matrix of an aryl-containing polymer, or by coating or encapsulating such an aromatic water-soluble active principle in a shell formed from such an aryl-containing polymer.

Another subject of the present invention is a composition, in particular a cosmetic composition, containing the particles described above.

The active principles stabilized by incorporation into an aryl-containing polymer matrix or shell according to the invention are chosen from aromatic water-soluble active principles of low molecular weight, i.e. less than 1000 and preferably less than 500. These active principles are preferably water-soluble.

According to the invention, the term “water-soluble active principle” means an active principle having a solubility in water, measured at 25° C., of at least 0.1 g/l (production of a macroscopically isotropic and transparent, coloured or uncoloured solution). This solubility is preferably greater than or equal to 1 g/l.

The hydrophilic nature of the aromatic active principles may be provided by one or more ionizable or ionized substituents, such as sulphonate, sulphate, carboxylate, phosphonate, phosphate, primary, secondary or tertiary amine groups or an ammonium group.

The active principles are preferably cosmetic active principles.

Examples of such cosmetic active principles that may be mentioned include:

hair dyes such as direct dyes and oxidation dye precursors,

UV-A and UV-B screening agents,

vitamins comprising an aromatic ring, such as vitamin B12,

flavonoids found in rye extracts,

aromatic reducing agents.

The term “synthetic polymers containing aryl groups” used to denote the polymers used in the manufacture of the particles of the present invention encompasses polymers comprising a number of aryl and/or arylene groups, preferably phenyl and phenylene groups, these groups possibly forming the main chain of the polymer or forming part of the side chains. They may be homopolymers or block, random or alternating copolymers, obtained by polyaddition or by polycondensation. This term also encompasses grafting polymers with a branched or crosslinked structure.

The aryl-containing polymers forming the matrix or shell of the particles of the present invention should, of course, be insoluble in water and in most cosmetically acceptable solvents. The aryl-containing polymers used in the present invention are consequently preferably nonionic, i.e. free of charges liable to make them soluble in water or in other polar solvents.

As indicated above, the glass transition temperature of the polymer forming the matrix or shell of the particles according to the invention should be at least equal to 45° C. It is preferably greater than or equal to 50° C. and ideally greater than or equal to 600° C. This parameter is essential for the invention. Indeed, it has been found that the set, vitreous nature of the polymer at room temperature is an important condition to prevent the migration of the encapsulated active principle molecules towards the surface of the particles, or the penetration of chemical agents into the particles. However, the particles of the present invention and the compositions containing them are liable to be exposed to relatively high temperatures prevailing, for example, in summer on a beach. When the temperature of the particles then approaches the glass transition temperature of the polymer, the polymer becomes plastic and there is a risk of leakage of the hydrophilic active principle out of the particles.

One group of aryl-containing polymers that is preferred according to the invention is formed by polyorganosiloxanes comprising aryl groups that are borne directly by the silicon atoms of the siloxane skeleton. The aryl-containing polyorganosiloxanes may have a linear, branched or crosslinked structure.

The appropriate content of aryl groups, and in particular phenyl groups, in the polyorganosiloxanes depends, inter alia, on the amount of active principle to be incorporated. Indeed, the inventors have found that for a given type of polymer, the amount of active principle that it is possible to encapsulate in a stable manner increases as the content of aryl groups increases. Moreover, the content of aryl groups has an influence on the glass transition temperature, which is generally higher for higher contents of aryl groups.

The inventors have obtained satisfactory results for an aryl group content such that the ratio of the number of aryl groups to the number of silicon atoms is between 1/15 and 2/1. Polyorganosiloxanes with a ratio of the number of aryl groups to the number of silicon atoms in the range from 1/10 to 2/1 are particularly preferred.

Examples of such phenyl-containing polyorganosiloxanes that may be mentioned include the polymer DC®-6018 sold by the company Dow Corning.

Another family of aryl-containing polymers that may be used to stabilize water-soluble aromatic active principles encompasses the polycondensates obtained from aryl-containing monomers.

These polycondensates are, for example, polyurethanes obtained by polycondensation of at least one diisocyanate and of at least one compound containing two functions containing labile hydrogen, chosen from hydroxyl, thiol, primary amine and secondary amine functions, the monomers being chosen such that at least one type of comonomer comprises an aryl group.

It emerges from the text hereinabove that the term “polyurethanes” as used in the present application encompasses not only polyurethanes per se comprising carbamate bonds (—NH—CO—O) but also polyureas (containing —NH—CO—NH— or —NR—CO—NH— bonds) and polythioureas (containing —NH—CO—S— bonds).

The aryl-containing polycondensates may also be polyesters or polyamides obtained by polycondensation of at least one diacid or one activated derivative of a diacid, with, respectively, at least one diol or at least one diamine, these monomers being chosen, as previously, such that at least one of the types of monomer comprises an aryl group.

The monomers giving the above aryl-containing polycondensates, i.e. polyurethanes, polyesters, polyamides and copolymers thereof, are known in the art.

Examples of non-aryl-containing monomers that may be mentioned include:

aliphatic diisocyanates such as hexamethylene diisocyanate and isophorone diisocyanate,

aliphatic diols such as alkylene glycols or polyalkylene glycols,

aliphatic diamines such as alkylene diamines,

aliphatic diacids such as succinic acid, fumaric acid, adipic acid and sebacic acid, or the anhydrides or chlorides of these diacids,

amino alcohols,

amino acids.

Examples of aryl-containing monomers that may be mentioned include:

aromatic diisocyanates such as toluene diisocyanate and diphenylmethane diisocyanate,

aromatic diamines such as phenylenediamines, which are optionally substituted,

aromatic diols such as bisphenols, for example bisphenol A, aromatic diacids such as terephthalic acid and isophthalic acid.

The polycondensation of the divalent monomers listed above results in linear structures. To obtain branched structures, a certain fraction of at least trivalent monomers may be added, such as 1,3,5-benzenetricarboxylic acid trichloride, silicon tetraisocyanate, 2,4,6-triamino-1,3,5-triazine, glycerol, polyglycerol or polypropoxylated glycerol containing amino end groups.

In one preferred embodiment of the invention, at least one type of monomer forming the aryl-containing polycondensates mentioned above (polyurethanes, polyesters and polyamides) comprises an S—S bond. This monomer may be different from that or those comprising an aryl group or may be identical thereto, in other words it may comprise both an aryl group and an S—S bond.

As examples of monomers comprising an S—S bond or both an aryl group and an S—S bond, mention may be made, for example, of:

bis(4-aminophenyl) disulphide, homocystine, cystamine, formamidine disulphide, bis(2-hydroxyethyl) disulphide.

Polymers containing S—S bonds are particularly advantageous in cases in which the use of the active principle necessitates the release of this principle into the surrounding medium. Such a release may then take place via cleavage of the S—S bonds of the aryl-containing polymers, with the aid of a suitable chemical agent.

The particles of the present invention may have very variable structures and sizes, resulting from the process for preparing them and linked to the intended use.

They may be, for example, microparticles obtained by grinding a solid material consisting of an aryl-containing polymer matrix in which the aromatic active principle(s) is (are) finely dispersed or dissolved (solid solution). These microparticles preferably have a mean size of between 0.5 and 500 μm.

Other microparticles with a size of between 0.05 and 500 μm may be obtained by multiple emulsion. They have a structure of core-shell type in which a shell formed by the aryl-containing polymer hermetically surrounds a liquid core containing the hydrophilic aromatic active principle. The multiple emulsion technique used for the formation of particles of this type is described, for example, in FR 2 766 737 and in DE 19719297.

In another embodiment, the particles are particles with a core-shell structure that are relatively larger in size, preferably between 0.5 and 10 mm. These particles are obtained by coextrusion of two phases, the outer phase being formed by the aryl-containing polymer, in melt form or in the form of a viscous solution, preferably with a viscosity (measured at 25° C.) of greater than 1000 centipoises, and the inner phase containing the active principle in melt form or in the form of a thick solution.

To effectively protect the active principle in the polymer matrix of the particles of the invention, it is preferable for the weight ratio of the active principle to the aryl-containing polymer not to exceed a certain limiting value that depends, for example, on the content of aryl groups of the polymer. The higher this content, the higher the upper limit of the active principle/aryl-containing polymer weight ratio giving rise to effective protection. The inventors have found that satisfactory encapsulation results are generally obtained for a weight ratio of the aromatic water-soluble active principle to the aryl-containing polymer of between 1/1 and 1/50 and preferably between 1/3 and 1/20.

As mentioned in the introduction, a subject of the present invention is also a process for protecting or stabilizing aromatic active principles. This process more precisely comprises the incorporation of an active principle, preferably a water-soluble active principle, comprising one or more aromatic, carbocyclic or heterocyclic, monocyclic or fused polycyclic groups, with a molecular weight of less than or equal to 1000, into a matrix of a synthetic polymer containing aryl groups with a glass transition temperature (T_{g}) of greater than or equal to 45° C., or the coating or encapsulation of such an active principle in a shell formed by such a polymer containing aryl groups.

The incorporation of the active principle into a matrix may be performed either by fusion or by dissolution of the polymer and the active principle.

The fusion process comprises the following steps:

melting the synthetic polymer containing aryl groups,

dissolving or finely dispersing the aromatic active principle in the molten polymer,

allowing the mixture thus obtained to cool to room temperature, and

grinding the solid material to a suitable particle size, preferably of between 0.05 and 500 μm.

This process has the advantage of being able to be performed in the absence of organic solvents. However, it has the difficulty of being able to be performed only with polymers whose melting point is less than the decomposition temperature of the active principle.

The dissolution process makes it possible to overcome this drawback. This process comprises:

dissolving the synthetic polymer containing aryl groups and the aromatic active principle in a suitable solvent or mixture of solvents,

evaporating off the solvent so as to obtain a solid material, and

grinding the solid material to a suitable particle size, preferably of between 0.05 and 500 μm.

This process requires the selection of a solvent or mixture of solvents that allows both the hydrophobic aryl polymer and the active principle, which is often hydrophilic, to be dissolved.

Examples of such solvents or mixtures of solvents that may be mentioned include C₁₋₄ alcohols such as methanol, ethanol and isopropanol. Other organic solvents such as DMSO and THF may also be used.

The grinding of the vitreous solid material obtained after cooling to room temperature or after evaporating off the solvent may be performed using any type of mill known in the art that makes it possible to obtain particles of the desired size, preferably between 0.05 and 500 μm. Examples that may be mentioned include knife mills, ball mills and pin mills.

Finally, according to a third embodiment, the process for protecting or stabilizing aromatic water-soluble active principles comprises coextrusion of the aromatic water-soluble active principle and of the synthetic polymer containing aryl groups so as to obtain particles consisting of a solid core formed by the aromatic water-soluble active principle, and of a solid shell formed by the synthetic polymer containing aryl groups.

The coextrusion of the aryl-containing polymer and of the aromatic water-soluble active principle is performed according to a known process described, for example, in Harper J. M., 1990, Extrusion of Foods in biotechnology and food process engineering, published by Marcel Deker Inc., Chapter 10, page 307.

A subject of the present invention is also compositions, preferably cosmetic compositions, comprising, preferably in a cosmetically or physiologically acceptable medium, the particles described above. The amount of particles present in the compositions of the present invention depends on the size of the particles, their content of active principle and, of course, the type of formulation and its intended use. In general, the compositions of the present invention comprise from 0.1% to 95% by weight and preferably from 1% to 50% by weight of particles relative to the total weight of the composition.

The cosmetically or physiologically acceptable medium should be inert with respect to the aryl-containing polymer used to prepare the particles, and in particular should neither dissolve nor swell this polymer.

The cosmetically or physiologically acceptable medium preferably contains a large fraction of water, and care should be taken to ensure that the concentration of certain organic solvents capable of dissolving the polymer, such as ethanol or glycols, does not exceed a certain value.

The compositions according to the present invention may also contain active principles and formulation ingredients of all kinds usually used in cosmetics.

The cosmetic active principles may be chosen from materials including vitamins or provitamins, saccharides, oligosaccharides and polysaccharides, which are hydrolysed or non-hydrolysed, and modified or unmodified, amino acids, oligopeptides, peptides and proteins, which are hydrolysed or non-hydrolysed, and modified or unmodified, amino polyacids, branched or unbranched fatty acids and fatty alcohols, fatty esters, animal, plant or mineral waxes, plant, mineral or synthetic oils, ceramides and pseudoceramides, hydroxylated organic acids, UV-screening agents, antidandruff agents, seborrhoea regulators, calmatives, cationic surfactants, cationic polymers, amphoteric polymers, organomodified or non-organomodified silicones, and associative polymers of nonionic, cationic or amphoteric type comprising at least one fatty chain.

The formulation adjuvants may be chosen, for example, from thickeners, agents for adjusting and fixing the pH, preserving agents, sequestering agents, opacifiers, reducing agents, antioxidants, fragrances and non-cationic surfactants.

Needless to say, a person skilled in the art will take care to preferably select the optional additional compound(s) above such that the intrinsic advantageous properties of the compositions according to the invention are not adversely affected by the envisaged addition(s).

The compositions of the present invention are preferably compositions for dyeing keratin fibres, and the stabilized active principles are then preferably chosen from hair dyes.

In another embodiment, the compositions of the present invention are antisun compositions and the encapsulated active principles are then organic UV-screening agents.

In yet another embodiment, the compositions of the present invention are self-tanning compositions and the encapsulated active principles are hydrophilic self-tanning active agents such as apigenidin.

The examples below demonstrate in particular the protective effect of the encapsulation of the aromatic hydrophilic active principles in an aryl-containing polymer matrix or shell.

### EXAMPLE 1

Preparation of Ground Particles

A phenylpropylsilsesquioxane of mass 1500-2500 g/mol, sold under the name DC® Z-6018 by the company Dow Corning (T_{g}=48° C.), is heated to a temperature of 130° C. so as to melt it, and 10% by weight, relative to the polysiloxane, of azo dye (MIP Red 2985-D, from Ciba) is dispersed therein until the mixture is homogeneous. After cooling to room temperature, the vitreous solid material obtained is ground using a mortar to a mean particle size of about 500 Am and the powder obtained is washed three times with water or with an aqueous solution of a dispersant (dimethicone copolyol isostearate) with vigorous agitation for 24 hours using a shaker (Prolabo). The purpose of this washing is to remove the dye molecules that are at the surface of the ground particles. An assay of the amount of dye contained in the particles thus washed reveals that 8% of the dye has been removed by washing, i.e. the degree of encapsulation is 92%.

The powder is then dispersed, using the above silicone dispersant, in an ammoniacal solution at pH 10.5 containing 0.53% by weight of sodium metabisulphite, and in an ammoniacal solution at pH 10.5 free of reducing agent.

After storing these two suspensions at a temperature of 45° C. for 2 months, the assay of the amount of dye that has leaked into the metabisulphite-free ammoniacal aqueous medium indicates a degree of encapsulation of 90% instead of the 92% initially measured, i.e. a loss of about 2% of the amount of dye initially encapsulated.

The assay of the amount of dye remaining in the particles stored for 2 months at 45° C. in the sodium metabisulphite solution, by UV-visible spectrometry of a solution of the powder in a chloroform/methanol mixture (9/1), reveals a loss of dye of about 5% (87% of dye remaining instead of the 92% initially encapsulated).

The fact that the loss of dye in the presence of reducing agent (5%) is slightly higher than that observed in the absence of reducing agent (2%) shows that a small fraction of encapsulated dye has been destroyed by the reducing agent. This result may be attributed to the very large specific surface area of the finely divided powder, which promotes exchanges between the particles and the surrounding medium.

### EXAMPLE 2

Encapsulation of Sorghum Extract

The third example relates to the encapsulation of sorghum extract, a complex dyestuff used especially for colouring the skin in self-tanning compositions. The majority of self-tanning compositions contain as main self-tanning active principle dihydroxyacetone (DHA), which is capable of chemically degrading apigenidin, which is a dye present in sorghum extract. It is thus a matter of protecting the sorghum extract, and in particular the apigenidin, against degradation by the dihydroxyacetone.

The encapsulation of the sorghum extract is performed in solvent medium. 1 part of sorghum extract and 10 parts of phenylpropylsilsesquioxane (DC® Z-6018, Dow Corning) are dissolved in a sufficient amount of ethanol. After total dissolution, the ethanol is evaporated off under vacuum and the solid residue is ground with a mortar to a mean particle size of about 500 μm. These particles are washed in the manner described in Example 2 in an aqueous solution.

To evaluate the degree of encapsulation after washing, the apigenidin after dissolution of the powder in acidic methanol (containing 0.1% by volume of 36% HCl) is assayed by UV-visible spectrometry. The degree of encapsulation after washing thus measured is between 90% and 95%.

To evaluate the efficacy of protection of the sorghum extract against chemical degradation by the DHA, an aqueous suspension, gelled with hydroxypropylcellulose, containing 2% of powder and 4% of DHA, and also an aqueous suspension, gelled with hydroxypropylcellulose, containing 0.2% of sorghum extract (non-encapsulated) and 4% of DHA, are prepared.

After storing these two samples at room temperature for 1 month, the level of degradation of the sorghum extract is 10% by weight for the encapsulated sample and 35% by weight for the non-encapsulated sample.

The above written description of the invention provides a manner and process of making and using it such that any person skilled in this art is enabled to make and use the same, this enablement being provided in particular for the subject matter of the appended claims, which make up a part of the original description.

As used above, the phrases “selected from the group consisting of,” “chosen from,” and the like include mixtures of the specified materials.

All references, patents, applications, tests, standards, documents, publications, brochures, texts, articles, etc. mentioned herein are incorporated herein by reference. Where a numerical limit or range is stated, all values and subranges therewithin are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. Particles comprising:
at least one active principle comprising one or more aromatic, carbocyclic or heterocyclic, monocyclic or fused polycyclic groups, with a molecular weight of less than or equal to 1000, and
at least one synthetic polymer comprising aryl groups, with a glass transition temperature (T_{g}) of greater than or equal to 45° C.

2. The particles according to claim 1, wherein the active principle(s) is (are) water-soluble.

3. The particles according to claim 1, wherein the aryl groups of the synthetic polymer are phenyl or phenylene groups.

4. The particles according to claim 1, wherein the aromatic active principle has a molecular weight of less than or equal to 500.

5. The particles according to claim 1, wherein the aromatic active principle bears at least one ionized or ionizable group.

6. The particles according to claim 1, wherein the weight ratio of the active principle to the aryl-containing polymer is 1/1-1/50.

7. The particles according to claim 1, wherein the aromatic active principle is a cosmetic active principle.

8. The particles according to claim 5, wherein the cosmetic active principle is a hair dye, an organic UV-screening agent or a flavonoid.

9. The particles according to claim 1, wherein the synthetic polymer comprising aryl groups is a nonionic polymer.

10. The particles according to claim 1, wherein the polymer containing aryl groups is a polyorganosiloxane comprising aryl groups linked directly to the silicon atoms of the siloxane skeleton.

11. The particles according to claim 10, wherein the ratio of the number of aryl groups to the number of silicon atoms in the polymer is 1/15-2/1.

12. The particles according to claim 1, wherein the synthetic polymer containing phenyl groups is a polyurethane obtained by polycondensation of:
at least one diisocyanate, and
at least one compound comprising two functions comprising labile hydrogen, selected from the group consisting of hydroxyl, thiol, primary amine and secondary amine functions,
the monomers being chosen such that at least one type of monomer comprises an aryl group.

13. The particles according to claim 1, wherein the synthetic polymer comprising aryl groups is a polyester or polyamide obtained by polycondensation:
of at least one diacid or of one activated derivative of a diacid, and
respectively, of at least one diol or of at least one diamine,
the monomers being chosen such that at least one type of monomer comprises a phenyl group.

14. The particles according to claim 12, wherein at least one type of monomer comprises an S—S bond.

15. The particles according to claim 1, wherein they are microparticles with a mean size of 0.05-500 μm, obtained by grinding a solid material.

16. The particles according to claim 1, wherein they are microcapsules of core-shell structure with a mean size of between 0.05 and 500 μm, obtained by multiple emulsion.

17. The particles according to claim 1, wherein they are coated particles with a mean size of 0.5-10 mm, obtained by coextrusion.

18. A process for protecting or stabilizing aromatic active principles comprising incorporating a water-soluble active principle comprising one or more aromatic, carbocyclic or heterocyclic, monocyclic or fused polycyclic groups, with a molecular weight of less than or equal to 1000, into a matrix of a synthetic polymer comprising aryl groups with a glass transition temperature (T_{g}) of greater than or equal to 45° C., or coating or encapsulating an active principle in a shell formed by such a polymer containing aryl groups.

19. The process according to claim 18, wherein it comprises:
melting the synthetic polymer containing aryl groups,
dissolving or finely dispersing the aromatic active principle in the molten polymer,
allowing the mixture thus obtained to cool to room temperature, and
grinding the solid material to a suitable particle size.

20. The process according to claim 18, wherein it comprises:
dissolving the synthetic polymer containing aryl groups and the aromatic active principle in a solvent or mixture of solvents,
evaporating off the solvent so as to obtain a solid material, and
grinding the solid material to a suitable particle size.

21. The process according to claim 18, wherein it further comprises a step of forming a multiple emulsion.

22. The process according to claim 18, wherein the aromatic active principle and the synthetic polymer comprising aryl groups are co-extruded so as to obtain particles comprising a solid core formed by the aromatic water-soluble active principle, and of a solid shell formed by the synthetic polymer comprising aryl groups.

23. A composition comprising, in a cosmetically or physiologically acceptable medium, particles according to claim 1.

24. The composition according to claim 23, wherein the particles represent 0.1%-95% by weight of the total composition.

25. The composition according to claim 23, wherein the particles represent 1%-50% by weight of the total composition.

26. The particles according to claim 1, wherein the weight ratio of the active principle to the aryl-containing polymer is 1/3-1/20.
